(19) United States
(12) Patent Application Publication
(10) Pub. No.: US 2012/0296188 A1
(43) Pub. Date: Nov. 22, 2012

(54) DEVICES AND METHODS FOR MEASURING OXYGEN

(75) Inventors: PERIANNAN KUPPUSAMY, NEW ALBANY, OH (US); BRIAN RIVERA, COLUMBUS, OH (US); EDWARD ETESHOLA, GAHANNA, OH (US); GURUGUHAN MEENAKSHISUNDARAM, BEACHWOOD, OH (US)

(73) Assignee: THE OHIO STATE UNIVERSITY RESEARCH FOUNDATION, COLUMBUS, OH (US)

(21) Appl. No.: 13/473,269

(22) Filed: May 16, 2012

Related U.S. Application Data
(60) Provisional application No. 61/486,519, filed on May 16, 2011.

(51) Int. Cl. A61B 5/055; A61B 5/05

(52) U.S. Cl. 600/364

(57) ABSTRACT

A sensor for measuring oxygen concentration in a tissue or an organ of a subject is provided. The sensor includes a sensory element comprising at least one paramagnetic spin probe compound encapsulated in a biocompatible oxygen permeable material. A barrier layer partially covers the sensory element and is comprised of at least one biocompatible oxygen impermeable material. Oxygen concentration data may be acquired by applying the sensor to the tissue or the organ of the subject, and subsequently applying a magnetic resonance spectroscopy or imaging technique.

### CROSS-REFERENCE TO RELATED APPLICATIONS

This application claims priority to and any other benefit of U.S. Provisional Patent Application Ser. No. 61/486,519, filed on May 16, 2011, the content of which is hereby incorporated by reference.

### STATEMENT ON FEDERALLY FUNDED RESEARCH

This invention was funded at least in part by a grant from the National Institutes of Health (NIH EB004031). The government may have certain rights in this invention.

### TECHNICAL FIELD

The present disclosure relates to oxygen measurements, and more particularly to devices and methods for measuring oxygen concentration in a tissue or an organ of a subject.

### BACKGROUND

The ability to measure oxygen concentration in living systems (in vivo) is an important clinical tool. The oxygenation level of tissue and organs serves as a predictor of tissue and organ viability. A patient with a wound, tissue, or organ that is poorly oxygenated is predisposed to tissue necrosis and potentially life-threatening infection because oxygen is used as an energy source for cells and as a substrate to mediate cell signaling and bacterial killing. Thus, knowledge of tissue and organ oxygenation levels provides clinicians with information that is both diagnostic and prognostic, especially in the field of wound healing. For example, tissue oxygenation levels help clinicians determine which tissues are viable, which tissues are threatened, but salvageable, and which tissues are unrecoverable or not viable, which allows the clinician to make better informed decisions in managing a patient.

The inability to distinguish between tissues and organs that have an adequate oxygenation level to heal or survive and those that do not has significant consequences for the patient and a huge impact on the cost of health care. Patients frequently must undergo a series of operations to inspect the site of tissue injury or infarction to remove necrotic tissue (a procedure called debridement) before any attempts are made to fix an underlying fracture, connect two ends of resected bowel or intestine, or cover a wound with a flap or graft to close a defect. Failure to adequately remove necrotic tissue is the leading cause of flap or graft failure and predisposes the site to infection, and can also cause other complications such as osteomyelitis, failure of fractures to heal, peritonitis (severe intra-abdominal inflammation), and surgical site incisions that must be left open to heal using a prolonged course of dressing changes.

Currently, there are only two technologies in clinical use that directly measure oxygenation status: arterial catheters and transcutaneous oxygen electrodes. These technologies are primarily used for critical-care monitoring. Arterial catheters are most commonly placed and maintained within the radial artery to measure oxygen levels in the blood that indicate systemic oxygen availability and not specific tissue oxygenation. This is an important distinction because adequate oxygenation of the blood is not always accompanied by tissue uptake of oxygen. The invasive nature of monitoring oxygenation levels with a catheter placed within a blood vessel is also accompanied by significant risks to the patient such as direct access of bacteria from the external environment to the patient's blood stream along the surface of the catheter and occlusion of the artery by the catheter resulting in critical ischemia.

The transcutaneous oxygen monitor (TcOM) is the only non-invasive, clinically-approved means by which to obtain tissue oxygen perfusion data. The method is quantitative, and it is the only device that measures oxygen delivery to an end organ (the skin). It has been used to monitor oxygenation levels (in mmHg) in the skin, especially for premature infants, but also for adults in the intensive care setting. The TcOM technique is commonly used to determine the healing capacity of tissue, to select amputation level, to assess the severity of arterial blockage, to predict the outcome of revascularization procedures, and to assess the severity and progression of peripheral vascular disease. In the TcOM procedure, TcOM electrodes are attached to self-adhesive rings that are placed on the skin. The electrodes are kept on the skin for a period of time, during which heating elements within the electrodes are active, promoting dilation of the underlying capillaries. The sensors then measure the oxygen diffusing through the skin.

Unfortunately, current TcOM technology has significant limitations. For example, dilation of the blood vessels beneath the electrode during TcOM lead heating may falsely elevate or represent an idealized tissue oxygenation value. In addition, TcOM technology does not allow for repeated direct measurements of oxygen in the same tissue or cells on a temporal scale. Moreover, the TcOM lead does not work when placed directly in a wound. Instead, oxygen measurements must be obtained from intact skin adjacent to the wound. This method provides only an indirect assessment of wound oxygenation that may not be accurate because the blood supply to the intact skin may come from a different perforating blood vessel than the blood supply to the wound. Furthermore, it may be difficult or impossible to obtain oxygen measurements via TcOM in patients that are obese, have significant lower extremity edema, or thickened skin, which conditions are extremely common in patients with lower extremity wounds. Finally, to accurately perform transcutaneous oxygen measurements on lower extremity wounds, according to standard clinical protocol, takes at least thirty minutes to one hour to complete.

Thus, there remains a need in the art for improved methods and devices for measuring the tissue or organ oxygen concentration of a subject. Such methods and devices should have minimal or no invasiveness and the capability to make repeated measurements from the region of interest in order to follow the changes in oxygenation over a period of time, preferably for up to several weeks, months, or even years. Moreover, such methods and devices should be available for use in the clinical setting, and should have shorter measurement times.

### BRIEF SUMMARY

The present disclosure relates to devices and methods for measuring oxygen concentration in a tissue or an organ of a subject. In an exemplary embodiment, the device for measuring oxygen concentration in a tissue or an organ of a subject is a sensor. The sensor includes a sensory element comprising at least one paramagnetic spin probe compound encapsulated in a biocompatible oxygen permeable material. In certain embodiments, it may be desirable to use more than one material for the biocompatible oxygen permeable material. The sensor also includes a barrier layer partially covering the sensory element. The barrier layer comprises at least one biocompatible oxygen impermeable material. The sensory element has a sensory contact surface for contacting the tissue or the organ of the subject, and the barrier layer covers the outer surface of the sensory element except for the sensory contact surface.

In an exemplary embodiment, a method for measuring oxygen concentration in a tissue or an organ of a subject includes the steps of: a) applying a sensor of the present disclosure to the tissue or the organ of the subject; and b) applying a magnetic resonance spectroscopy or imaging technique to obtain data corresponding to the amount of oxygen present in the tissue or the organ of the subject. In a preferred embodiment, the magnetic resonance spectroscopy or imaging technique is electron paramagnetic resonance.

### BRIEF DESCRIPTION OF THE DRAWINGS

FIG. 1 shows an embodiment of a sensor for measuring oxygen concentration in a tissue or an organ of a subject from a bottom plan view (A), a top plan view (B), and a cross-sectional view (C).

FIG. 2 shows the chemical structure of polydimethylsiloxane (PDMS).

FIG. 3 shows the molecular structure (A) and microcrystals (B) of the paramagnetic spin probe compound lithium octa-n butoxynaphthalocyanine (LiNc-BuO) radical.

FIG. 4 shows (A) the effect of oxygen concentration (pO₂) on the EPR spectrum of LiNc-BuO, the linewidth increases linearly with pO₂ in the range 0 to 160 mmHg with a slope (oxygen sensitivity) of 8.50 mG/mmHg. (B) A perspective view of LiNc-BuO radical down the c-axis. A “ball and stick” representation of the structure is employed. The structure shows wide-open channels of cross-sectional dimensions 8.1-9 Å, facilitating diffusion of oxygen molecules in and out of the channels.

FIG. 5 shows (A) long-term monitoring of in situ pO₂ in the mouse heart. (B) Myocardial tissue pO₂ from mice (n=7) implanted with LiNc-BuO radical microcrystals in the mid-ventricular region is shown. Data show the feasibility of pO₂ measurements for more than 4 months after implantation.

FIG. 6 shows various embodiments of sensory elements fabricated by the encapsulation of LiNc-BuO radical microcrystals in PDMS. (A) Pure PDMS film without any paramagnetic spin probe compound. (B) A LiNc-BuO:PDMS sensory element fabricated with 40 mg of LiNc-BuO radical microcrystals in 5 g of PDMS. (C) LiNc-BuO:PDMS sensory elements with varying sizes, shapes and ratios of paramagnetic spin probe compound to polymer (top view) (D) Side view of (C). Images demonstrate the successful fabrication of LiNc-BuO:PDMS sensory elements in different shapes and sizes, with different thicknesses and varying amounts of paramagnetic spin probe compound (LiNc-BuO radical microcrystals).

FIG. 7 shows LiNc-BuO:PDMS sensory elements with increasing spin density. Four different formulations of LiNc-BuO:PDMS, viz. C-5, C-10, C-20, and C-40, were fabricated by incorporating 5, 10, 20 and 40 mg of LiNc-BuO radical microcrystals, respectively, in the same amount of PDMS (5 g). Spin density of the LiNc-BuO:PDMS sensory element formulations are shown. Spin density was evaluated using a pre-calibrated standard, at X-band (9.8 GHz). Results (mean±SD, n=3), normalized by sample weight, show a linear relationship between the mass of LiNc-BuO radical microcrystals incorporated and the spin density of the four sensory element formulations.

FIG. 8 shows X-band EPR images of LiNc-BuO:PDMS sensory element formulations. Distribution of spins was evaluated using X-band EPR imaging. Samples were imaged under anoxic conditions, in a sealed tube. Image intensity correlated directly with the normalized spin density results shown in FIG. 13. Images demonstrate a high-degree of uniformity in the distribution of LiNc-BuO radical spins within the PDMS matrix in all four sensory element formulations.

FIG. 9 shows the oxygen response of a LiNc-BuO:PDMS sensory element. Oxygen calibration curves were constructed using peak-to-peak EPR linewidths of uncoated LiNc-BuO radical microcrystals and a sensory element at different levels of pO₂ (0-160 mmHg). The plot shows a linear relationship between linewidth and pO₂ for both uncoated LiNc-BuO radical and the sensory element, which was reversible and reproducible.

FIG. 10 shows the effect of sterilization. Sensory elements were sterilized by autoclaving and oxygen-calibration was determined. The data show that the oxygen calibration of the sensory element remained intact after autoclave sterilization.

FIG. 11 shows the effect of gamma irradiation. Sensory elements were irradiated with ⁶⁰Co-gamma radiation at doses of 15 and 30 Gy. EPR spin density and oxygen-calibration were determined before and after irradiation. The data show that irradiation has no significant effect on spin density or oxygen calibration of the sensory element.

FIG. 12 shows long-term stability and response of a sensory element to oxygen concentration, in vivo. The stability of the sensory element (1×1 mm²) implanted in the subcutaneous tissue (upper hind leg) of C3H mice was monitored for up to 60 days. The plot shows repeated measurements of pO₂ from a group of mice (group of symbols on top). The response of the sensory element to oxygen was checked by temporarily constricting blood-flow to the leg (group of symbols on bottom). The data show that the LiNc/PDMS sensory elements are stable and responsive in the tissue.

FIG. 13 shows the stability of EPR intensity (detection sensitivity) and oxygen response for continuous monitoring of pO₂. The measurements were performed using an LiNc-BuO:PDMS sensory element under room air and low-oxygen conditions continuously for more than 6 hours. The data show that both the EPR signal intensity and pO₂ measurements are stable.

FIG. 14 shows the temporal oxygen concentration response profile of a sensory element directly applied to the right hand of a human subject.

FIG. 15 shows the response of a sensory element to changes in oxygen concentration caused by constricting blood flow to the hand of a human subject having the sensory element directly applied to the hand. The data also shows the changes in oxygen concentration observed when the means for constricting blood flow to the hand was removed.

### DETAILED DESCRIPTION

The present invention will now be described by reference to more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the description and claims are to be understood as being modified in all instances by the term “about”. Accordingly, unless indicated to the contrary, the numerical parameters set forth in the following description and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this description will include every narrower numerical range that falls within such broader numerical range, as if such narrower numerical ranges were all expressly written herein.

Disclosed herein are devices and methods for measuring oxygen concentration in a tissue or an organ of a subject. As used herein, the term “oxygen concentration” refers to oxygen tension (pO₂) or oxygen partial pressure. For example, the devices and methods disclosed herein may be used to measure the partial pressure of oxygen diffusing through the skin of a human.

In an exemplary embodiment, the device is a sensor for measuring oxygen concentration in a tissue or an organ of a subject. The sensor comprises a sensory element comprising at least one paramagnetic spin probe compound encapsulated in a biocompatible oxygen permeable material. In certain embodiments, it may be desirable to use more than one material for the biocompatible oxygen permeable material. As used herein, the term “encapsulate” refers to a first material dispersed in a second material or materials, a first material within a matrix of a second material or materials, or a second material or materials doped with a first material. The sensor also includes a barrier layer partially covering the sensory element. The barrier layer comprises at least one biocompatible oxygen impermeable material. In certain embodiments, it may be desirable to use more than one biocompatible oxygen impermeable material for the barrier layer. Furthermore, in some embodiments, it may be desirable to have multiple barrier layers comprised of separate biocompatible oxygen impermeable materials. To allow contact with the tissue or the organ of the subject, the sensory element has a sensory contact surface, and the barrier layer covers the outer surface of the sensory element except for the sensory contact surface.

In some embodiments, the at least one paramagnetic spin probe compound may comprise ligands, dilithium complexes, and lithium radicals. Some preferred dilithium complexes are shown as compounds [1]-[6]:

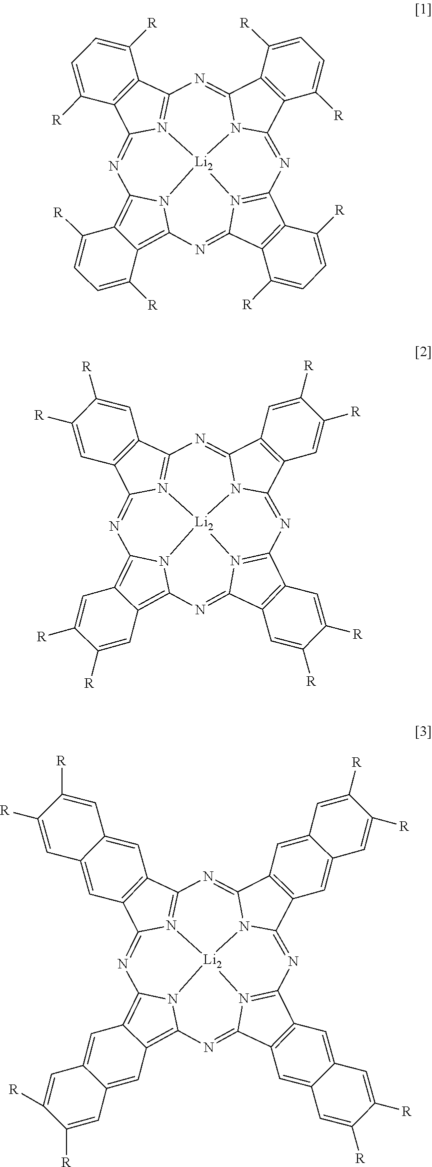

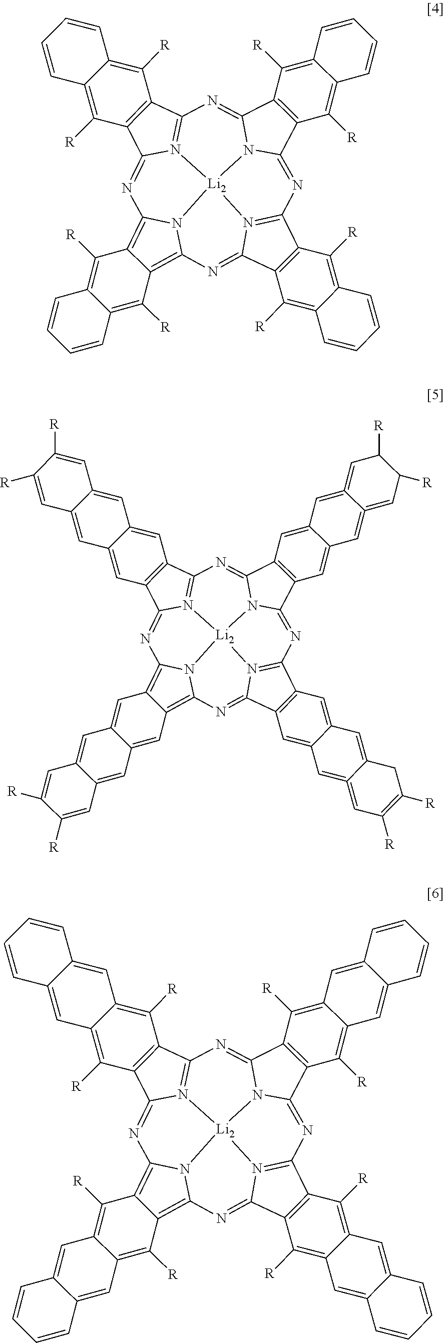

wherein R is selected from the group consisting of H, O(CH₂)ₙCH₃, S(CH₂)ₙCH₃, O(CH₂)ₙCH₂OH, O(CH₂)ₙCH₂NH₂, O(CH₂)ₙCH₂SH, and combinations thereof, wherein n is 1-6, and combinations thereof. Methods for synthesizing the paramagnetic spin probe compounds [1]-[6] are known in the art and scientific literature, such as in U.S. Pat. No. 7,662,362, which is incorporated by reference in its entirety.

For example, the following scheme shows the synthesis of paramagnetic spin probe compound [4].

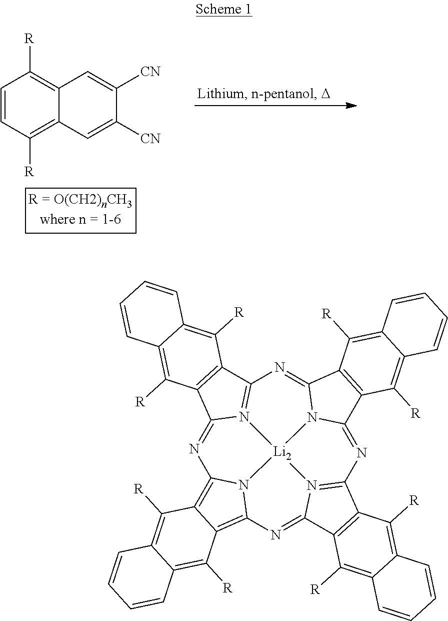

The at least one paramagnetic spin probe compound may also comprise lithium radicals. These lithium radicals may be synthesized by chemical or electrochemical oxidation of the dilithium complexes (compounds [1]-[6]). Such chemical and electrochemical oxidation techniques will be readily apparent to one of skill in the art. In a preferred embodiment, the at least one paramagnetic spin probe compound comprises lithium octa-n-butoxy-naphthalocyanine (LiNc-BuO) radical. The structure of this particular paramagnetic spin probe compound is shown below:

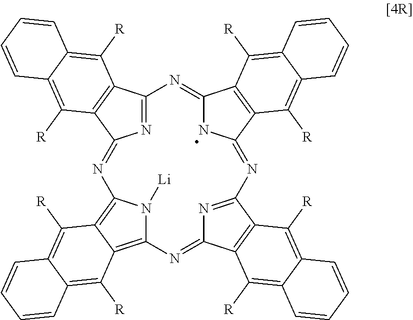

wherein R is O(CH₂)₃CH₃. A method for synthesizing compound [4R] is described in the literature and in U.S. Pat. No. 7,662,362. For example, lithium granules (0.0053 g, 0.774 mmol) are added to n-pentanol (15 ml) and refluxed for 30 min under nitrogen atmosphere. The mixture is cooled down to room temperature and 5,9,14,18,23,27,32,36-octa-n-butoxy-2,3-naphthalocyanine (0.1 g, 0.0774 mmol) is added and refluxed gently for 2.5 h under nitrogen atmosphere. After cooling down to room temperature, 300 ml of tert-butyl methyl ether is added and filtered through a small silica gel plug. The solvent is evaporated under reduced pressure to 3 ml of solution. The concentrate is dissolved in 100 ml of n-hexane. The greenish solution is slowly evaporated under reduced pressure to yield shiny crystals of lithium 5,9,14,18,23,27,32,36-octa-n-butoxy-2,3-naphthalocyanine (LiNc-BuO) radical. The crystals are washed with methanol and dried under vacuum.

Although several paramagnetic spin probe compounds are specifically described herein, those with skill in the art will appreciate that various other paramagnetic spin probe compounds may be utilized.

In some embodiments, the biocompatible oxygen permeable material or materials may be selected from the group consisting of polydimethylsiloxane, an amorphous fluoropolymer, fluorosilicone acrylate, cellulose acetate, polyvinyl acetate, and combinations thereof. In a preferred embodiment, the biocompatible oxygen permeable material is polydimethylsiloxane. In one embodiment, the amorphous fluoropolymer may be a random copolymer of tetrafluoroethylene and 2,2-bis((trifluoromethyl)-4,5-difluoro-1,3-dioxole, such as Teflon® AF from DuPont. Although this description specifically sets forth several biocompatible oxygen permeable materials, those of skill in the art will recognize that other biocompatible oxygen permeable materials may be used.

The amount of the at least one paramagnetic spin probe compound encapsulated in the biocompatible oxygen permeable material may vary widely. For example, in certain embodiments, the weight ratio of paramagnetic spin probe compound to biocompatible oxygen permeable material may be within a range of 1:1000 to 1:125. In another embodiment, the weight ratio of paramagnetic spin probe compound to biocompatible oxygen permeable material may be within a range of 1:500 to 1:250. In one embodiment, the sensory element may comprise 5 milligrams of paramagnetic spin probe compound and 5 grams of biocompatible oxygen permeable material. In another embodiment, the sensory element may comprise 10 milligrams of paramagnetic spin probe compound and about 5 grams of biocompatible oxygen permeable material. In still another embodiment, the sensory element may comprise 20 milligrams of paramagnetic spin probe compound and 5 grams of biocompatible oxygen permeable material. In yet another embodiment, the sensory element may comprise about 40 milligrams of paramagnetic spin probe compound and 5 grams of biocompatible oxygen permeable material. As will be explained below, the amount of paramagnetic spin probe compound encapsulated within the biocompatible oxygen permeable material may vary, yet still allow the sensor to provide accurate oxygen concentration measurements.

In certain embodiments, the at least one biocompatible oxygen impermeable material may be selected from the group consisting of polyvinyl alcohol, a poly(p-xylylene) polymer, an aluminum oxide-coated polyester film, a polyacrylic acid-coated polyester film, ethylene-vinyl alcohol, and combinations thereof. In a preferred embodiment, the at least one biocompatible oxygen impermeable material is polyvinyl alcohol. Polyvinyl alcohol is known to be biocompatible and has been approved for use by the U.S. Food and Drug Administration in medical applications. In another embodiment, the at least one biocompatible oxygen impermeable material is a poly(p-xylylene) polymer, such as parylene-N and parylene-C, available from Specialty Coating Systems, Inc. of Indianapolis, Ind. The poly(p-xylylene) polymer may also be halogenated, as in parylene-C and parylene AF-4. Although this description specifically sets forth several biocompatible oxygen impermeable materials, those of skill in the art will recognize that other biocompatible oxygen impermeable materials may be used. Moreover, in certain embodiments, the at least one biocompatible oxygen impermeable material may be formed as a composite of at least two biocompatible oxygen impermeable materials or as a multilayered material comprising at least two biocompatible oxygen impermeable materials. In some embodiments, the multilayered material may further include a layer of nylon-6.

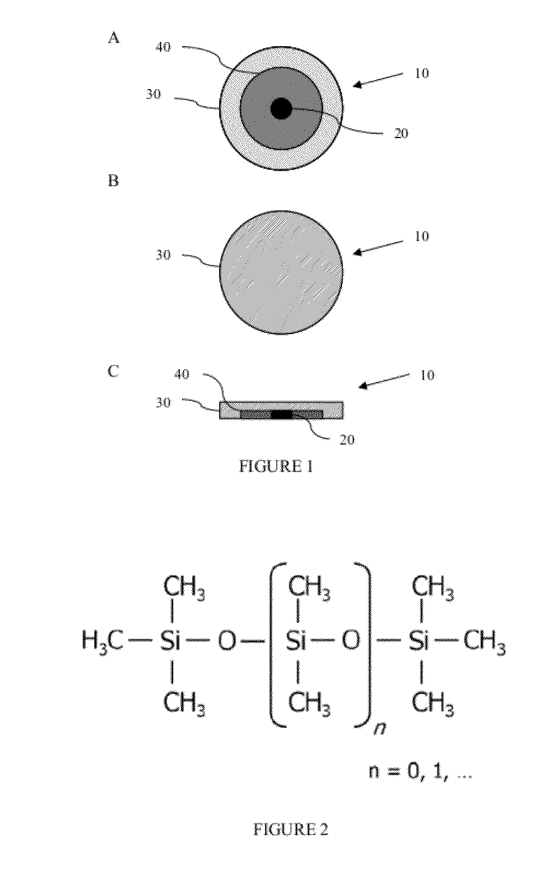

Referring now to FIG. 1, an exemplary embodiment of a sensor (10) for measuring oxygen concentration in a tissue or an organ of a subject is shown. As seen in FIG. 1, the sensor (10) includes a sensory element (20) and a barrier layer (30) partially covering the sensory element (20), as best seen in FIG. 1C. In one embodiment, the sensor element (20) may further include an outer protective layer (40) comprised of at least one biocompatible oxygen permeable material. The outer protective layer (40) ensures that the at least one paramagnetic spin probe compound itself does not come into direct contact with the tissue or the organ of the subject. The outer protective layer (40) may comprise a biocompatible oxygen permeable material that is the same or different from the biocompatible oxygen permeable material used to encapsulate the at least one paramagnetic spin probe compound. In a preferred embodiment, the outer protective layer (40) is polydimethylsiloxane.

In one embodiment, the sensor (10) may consist of a sensory element (20) encapsulated by an outer protective layer (40). This particular embodiment may be useful for implanting the sensor (10) in a tissue or an organ of a subject. As noted above, the outer protective layer (40) comprises a biocompatible oxygen permeable material, such as polydimethylsiloxane. Again, in this embodiment, the sensor (10) may be implanted to temporally monitor the oxygen concentration of deep tissues or internal organs of a subject.

Although the sensor (10) shown in FIG. 1 is formed as a disk, various other shapes may be used. For example, the sensor (10) may be formed as a thin film of any desired shape, a cube, a sphere, a rectangular prism, a cone, or a pyramid, just to name a few. Moreover, while the sensory element (20) is depicted as having a flat sensory contact surface, the sensory contact surface may be formed with a contour to conform to virtually any tissue or organ surface.

In addition to various shapes, the sensor (10) and the materials comprising the sensor (10) may have varying thicknesses and sizes. For example, in some embodiments, the sensor (10) may have a thickness of about 0.5 millimeter to about 1 centimeter, and a width or length of about 0.5 millimeter to about 1 centimeter. In some embodiments, the sensory element (20) may have a thickness of about 0.25 millimeter to about 5 millimeter, and the barrier layer (30) may have a thickness within the range of about 0.25 millimeter to about 1 centimeter. With respect to the barrier layer (30), those with skill in the art will appreciate that the thickness of the barrier layer (30) may depend on the specific type of material utilized to make the barrier layer (30) substantially impermeable to oxygen. In a preferred embodiment, the sensor (10) is disk shaped and has a diameter of about 5 millimeters and a thickness of about 1 millimeter, with the sensory element (20) having a diameter and a thickness of about 0.5 millimeter and the barrier layer (30) having an inside diameter of about 0.5 millimeter, an outside diameter of about 5 millimeters, and a thickness within the range of about 0.5 millimeter to about 1 millimeter. In another embodiment, the sensor (10) is disk shaped and has a diameter of about 5 millimeters and a thickness of about 1 millimeter, with the sensory element (20) having a diameter and a thickness of about 0.5 millimeter, the barrier layer (30) having an inside diameter of about 2 millimeters, an outside diameter of about 5 millimeters, and a thickness within the range of about 0.5 millimeter to about 1 millimeter, and an outer protective layer (40) having an inside diameter of about 0.5 millimeter, an outside diameter of about 2 millimeters, and a thickness of about 0.5 millimeter.

In an exemplary embodiment, the sensor may include a biocompatible adhesive layer to allow the sensor to stick to a tissue or an organ of a subject. In one embodiment, the adhesive layer is provided on at least a portion of the outer surface of the barrier layer. For example, the adhesive layer may be provided on a portion of the outer tissue or organ contacting surface of the barrier layer. Preferably, the adhesive layer is biocompatible, such as 2-octyl cyanoacrylate. In other embodiments, the adhesive layer is biocompatible and oxygen impermeable.

In an exemplary embodiment, a method of measuring oxygen concentration in a tissue or an organ of a subject comprises the steps of: a) applying a sensor, as described herein, to the tissue or the organ of the subject; and b) applying a magnetic resonance spectroscopy or imaging technique to obtain data corresponding to the concentration of oxygen present in the tissue or the organ of the subject. Applicable magnetic resonance spectroscopy or imaging techniques include, but are not limited to, electron paramagnetic resonance (EPR), electron spin resonance (ESR), electron paramagnetic resonance imaging (EPRI), magnetic resonance imaging (MRI), and proton-electron double-resonance imaging (PEDRI). In a preferred embodiment, the magnetic resonance spectroscopy or imaging technique applied is electron paramagnetic resonance (EPR).

The electron paramagnetic resonance (EPR) spectroscopy technique has been utilized to measure oxygen concentration, and the process is commonly referred to as EPR oximetry. The principle of EPR oximetry is based on the paramagnetic characteristics of molecular oxygen, which in its ground state has two unpaired electrons, and undergoes spin exchange interaction with a paramagnetic spin probe compound. This process is sensitive to the amount of oxygen present in the local environment, with the relaxation rate of the paramagnetic spin probe compound increasing as a function of oxygen content (i.e., concentration or partial pressure). The increased spin-spin relaxation rate results in increased line-broadening. The fact that the linewidths of EPR resonance lines correlate with oxygen concentration has been used in a variety of biological settings. The development of low frequency EPR instrumentation at L-band (1-2 GHz) and even lower frequencies (600 MHz and 300 MHz) has made it possible to perform EPR oximetry measurements on complex biological systems such as intact animals and isolated functioning organs.

In an exemplary embodiment, the method of measuring oxygen concentration is used to measure transcutaneous oxygen levels of a human subject. In this embodiment, the tissue or the organ of the subject is the skin, and the sensor is directly applied to the skin of the subject. For example, a sensor may be directly applied to the hand of a human subject. Next, the human subject may place their hand with the sensor applied thereto under the resonator of a commercially-available L-band EPR unit so that data corresponding to the concentration of oxygen present in the skin at the location of the sensor may be obtained.

In one embodiment, the method of measuring oxygen concentration may further include the step of waiting a predetermined amount of time before applying the magnetic resonance spectroscopy or imaging technique to obtain oxygen concentration data. For example, in certain embodiments, after the sensor is applied to the tissue or the organ of the subject, a period of from about 5 minutes to about 90 minutes is allowed to pass before the magnetic resonance spectroscopy or imaging technique is applied to obtain oxygen concentration data. This step will help ensure that the sensor and the tissue or the organ reach a state of equilibrium, and that the oxygen concentration data is not inaccurately reported due to oxygen being entrapped during sensor application.

The sensory element of the sensor may be fabricated by various methods, including but not limited to, cast-molding or injection-molding methods using a biocompatible oxygen permeable material doped with microcrystals of at least one paramagnetic spin probe compound. However, those with skill in the art will appreciate that other fabrication techniques may be employed. In certain embodiments, the microcrystals of the at least one paramagnetic spin probe compound have a size of 10 microns or less. In a preferred embodiment, the biocompatible oxygen permeable material is polydimethylsiloxane (PDMS) and the at least one paramagnetic spin probe compound is lithium octa-n-butoxy-naphthalocyanine (LiNc-BuO) radical (i.e., compound [4R], wherein R is O(CH₂)₃CH₃. The LiNc-BuO radical is particularly useful because published data has shown that normally-perfused human skin produces transcutaneous oxygen measurements on the order of about 50 mmHg to about 90 mmHg, and the sensitivity of LiNc-BuO radical is about 8.5 mG/mmHg, which is ideal for normoxic and hyperoxic applications. In clinical cases where hypoxic conditions (e.g., less than 30 mmHg) may be expected, it may be possible to use a different paramagnetic spin probe compound, such as lithium naphthalocyanine (LiNc) radical (i.e., compound [4R], wherein R is H), which has a sensitivity of 34 mG/mmHg and would provide a means by which to detect relatively small changes in tissue oxygen perfusion. Both LiNc-BuO radical and LiNc radical paramagnetic spin probe compounds are nonsaturable at X-band microwave powers of less than 25 mW.

As noted above, polydimethylsiloxane (PDMS) is a preferred biocompatible oxygen permeable material that may be used to construct the inventive sensor. PDMS is a flexible, optically clear, chemically and magnetically inert, non-toxic, non-flammable, hypoallergenic, and most importantly, intrinsically oxygen-permeable siloxane-based elastomeric polymer. PDMS is a synthetic polymer with an unusual molecular structure—a large backbone of alternating silicon and oxygen atoms. In addition to their links to oxygen to form the polymeric chain, the silicon atoms are also bonded to organic moieties, typically methyl groups. The chemical structure of PDMS is shown in FIG. 2. The unique properties of PDMS are due to the simultaneous presence of organic groups attached to an inorganic backbone that has been successfully exploited for fabrication into various sizes and shapes for medical devices used in contact with human tissue and body fluids for several decades. The toxicology of PDMS has been studied thoroughly because of its use in medicine and biomedical technology, as well as in pharmaceuticals and cosmetics. The innocuousness of siloxanes explains their numerous applications where prolonged contact with the human body is involved. Siloxane polymers are used in many approved medical devices regulated by the U.S. Food and Drug Administration and European Medical Devices Directive. The excellent biocompatibility and biodurability of siloxane polymers is partly due to low chemical reactivity, thermal stability, low surface energy and hydrophobicity. With respect to the present sensor, biocompatible oxygen permeable materials, such as PDMS, provide excellent gas permeability, which leads to increased oximetry sensitivity and consequent detection of lower oxygen tension levels in cells, tissues, or organs of a subject.

As previously noted, the barrier layer partially covers the sensory element, and comprises at least one biocompatible oxygen impermeable material. As its name suggests, the barrier layer effectively serves as a barrier to oxygen so that the sensor does not provide oxygen concentration data that is corrupted by local or ambient oxygen. Thus, in a preferred embodiment, the barrier layer covers all non-tissue-contacting surfaces of the sensor. A preferred biocompatible oxygen impermeable material used for the barrier layer is polyvinyl alcohol. However, as noted above, the barrier layer may comprise multiple biocompatible oxygen impermeable materials and/or multiple layers. In one embodiment, after the sensory element is fabricated, all but the basal surface of the sensory element may be coated with polyvinyl alcohol. During this coating process, the total diameter of the sensor will be increased, providing an outer rim or lip area that may be coated with an adhesive for attachment to a tissue or an organ.

The sensors described herein may be produced by various processes, including via a three-step process as discussed below. The liquid silicone injection molding (LSIM) and microinjection molding (LSMIM) fabrication methods offer many benefits in the fabrication of PDMS, including less expensive tooling, accurately molded parts, very fast and short heat cycles (which avoid the problem of flashing and material degradation), minimal material requirement and waste, and cleanliness. In these processes, pumping systems deliver the two-part liquid silicone (catalyst and crosslinker) directly into a mixer for homogenization and then directly into the mold cavity/die, in a completely closed process. Molding and curing occur rapidly within the mold cavity at a set temperature.

In this exemplary three step process, the first step may involve injection molding of the sensory portion of the chip. Dies may be produced for all three steps that can be used with an injection molding machine (Morgan Press G-55T by Morgan Industries; Long Beach, Calif.). In one embodiment, the first step may involve the preparation of the sensory element of the sensor by mixing the at least one paramagnetic spin probe compound with the biocompatible oxygen permeable material, heating the mixture, and forcing the mixture into a die. In certain embodiments, the at least one paramagnetic spin probe material and the biocompatible oxygen permeable material are mixed such that the at least one paramagnetic spin probe material is homogeneously distributed in the biocompatible oxygen permeable material. Upon cooling, the sensory element may be removed from the die and set aside.

In one embodiment, the second step may include producing an outer protective layer that surrounds the sensory element. However, as previously mentioned, certain embodiments do not include an outer protective layer. A separate die may be used to produce the outer protective layer. The process used to fabricate the sensory element may be replicated in this step, with one exception: no paramagnetic spin probe compound will be added to the biocompatible oxygen permeable material during the fabrication of the outer protective layer. After the outer protective layer has cooled, the outer protective layer may be removed from the die and set aside.

In one embodiment, a third and final step of the process may comprise adding the sensory element and outer protective layer into an injection mold die. Next, an overmolding process may be used to deposit the barrier layer on the sensory element and the outer protective layer. This step is important, as all non-tissue or organ contacting surfaces of the sensor must be covered by the barrier layer to prevent local or ambient oxygen from interfering with the tissue or organ oxygen concentration measurements. As previously noted, polyvinyl alcohol is a preferred biocompatible oxygen impermeable material for use as the barrier layer.

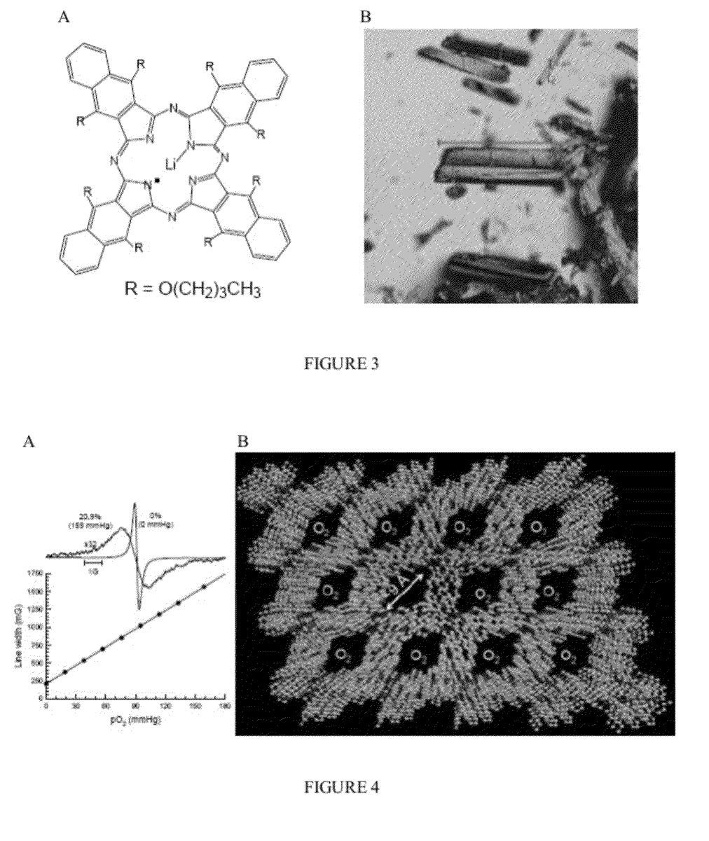

As mentioned above, the at least one paramagnetic spin probe compound may comprise lithium octa-n-butoxynaphthalocyanine (LiNc-BuO) radical microcrystals, as seen in FIG. 3. The LiNc-BuO radical microcrystals may be obtained by reacting lithium pentoxide with octa-n-butoxy-2,3-naphthalocyanine, which produces dark green crystals. The LiNc-BuO radical is insoluble in water, and is stable in air or in aqueous suspensions at ambient conditions. Moreover, the LiNc-BuO radical microcrystals exhibit a singleline EPR spectrum, with the peak-to-peak width of the spectrum being linearly dependent on the oxygen concentration, as seen in FIG. 4A. When exposed to biological oxido-reductants including superoxide, H₂O₂ (1 mM), NO, GSH (10 mM), or ascorbate (5 mM), or 15.5 Gy of Cobalt-60 γ-ray irradiation for 10 min, there is no effect on the EPR properties or oxygen sensitivity of the LiNc-BuO radical microcrystals (data not shown). Referring now to FIG. 4B, the 3D crystal structure of the LiNc-BuO radical shows the presence of wide-open channels through which oxygen can diffuse freely causing the observed effect of line-broadening. In addition, in order to evaluate the stability of the LiNc-BuO radical microcrystals in tissues, the LiNc-BuO radical microcrystals were implanted in the left-ventricular region of a mouse heart and repeated measurements of oxygen tension were performed in the same set of animals over a period of 120 days, as seen in FIG. 5. The results show that the LiNc-BuO radical compound has tissue stability for at least four months, and perhaps longer. In summary, the LiNc-BuO radical paramagnetic spin probe compound has a number of advantages, including, but not limited to, a single, sharp EPR spectrum, a linear variation of linewidth with oxygen concentration that is independent of the size of the LiNc-BuO radical microcrystals, and long-term stability in tissues.

The present inventive sensors have a number of advantages when compared to current TcOM technology. For example, as opposed to TcOM electrodes, the sensors disclosed herein will be significantly smaller and in the form of a thin film. Additionally, unlike TcOM sensors, the presently disclosed sensors will not require wiring leads. As previously mentioned, EPR spectroscopy may be used to obtain measurements of the partial pressure of oxygen (pO₂) diffusing through the tissue or the organ. As with TcOM, multiple sites may be covered with separate sensors to obtain oxygen concentration data. When using EPR spectroscopy, the oxygen concentration data collected will be highly repeatable and accurate. Importantly, the high degree of sensitivity of the paramagnetic spin probe compounds, such as the LiNc-BuO radical microcrystals, to the partial pressure of oxygen (pO₂) would permit detection without the need to heat the surrounding tissue or organ, a potential difficulty encountered when using TcOM. Moreover, the presently disclosed sensor may be utilized in an open wound bed to obtain oxygen concentration data that may be used to guide a clinician's decision in managing a patient. Furthermore, it may be possible to collect similar amounts of data in a shorter time period (expected to take less than two minutes per site) using the presently disclosed sensors, when compared to current TcOM technology.

The presently disclosed sensors provide a sterile, non-invasive method of measuring the oxygenation level for any tissue. However, in certain embodiments, the sensors may used invasively if desired. The sensors can provide clinicians with information about levels of tissue oxygenation directly in a wound or injured tissue in real-time with no delay, and the data obtained may even be transmitted wirelessly for use in telemedicine applications. The present sensors may be used to decrease mortality in critically ill patients. Moreover, the sensors may provide surgeons with quantitative measurements of oxygen concentration that can help guide debridement decisions to avoid removing too much tissue, which could decrease the success of reconstructive surgical procedures or compromise the function within the remaining organ tissue. Alternatively, the present sensors may help surgeons avoid taking too little, poorly-perfused tissue that could result in tissue necrosis and infection, as well as reducing the number of times a patient must be taken to the operating room for a surgical debridement. Still further, the sensors and the data provided thereby may decrease the incidence of surgical site infections (SSI) by decreasing the likelihood of leaving behind non-viable tissue. In addition, the present sensors may be used to monitor the viability of tissue flaps used to close defects due to trauma, cancerous tissue removal, or congenital causes, as well as identify early changes in oxygenation to diagnose a compromised flap. This can lead to an earlier return to the operating room to revise compromised flaps, which may result in an increased flap salvage rate.

The impact of the presently disclosed sensor on clinical care is vast. According to the Centers for Disease Control (CDC), a hospitalized patient that develops an SSI has a mortality that is more than double what it would be for that patient without a SSI. The incidence of SSIs is 14-16% among all hospitalized patients and 38% among surgical patients and a patient with an SSI spends an average of five additional days in the hospital. Oxygenation level has also played a critical role in limb salvage by aiding in the proper diagnosis and care of diabetic foot ulcers. There are over 23 million people in the US with diabetes, and 25% of people with diabetes will develop a foot ulcer over the course of their lifetime. Moreover, every year 1% of all people with diabetes will have an amputation. Amputations are twenty-eight times more common in patients with diabetes compared to those without diabetes, and limb salvage remains a high priority to avoid the subsequent disability associated with limb loss. The benefits of providing clinicians with tissue oxygenation information on a large scale basis has not been possible due to the limitations of existing technologies.

Having generally described the present sensor, a further understanding can be obtained by reference to certain specific examples illustrated below which are provided for purposes of illustration only and are not intended to be all inclusive or limiting unless otherwise specified.

### EXAMPLES

Sensory Element Fabrication:

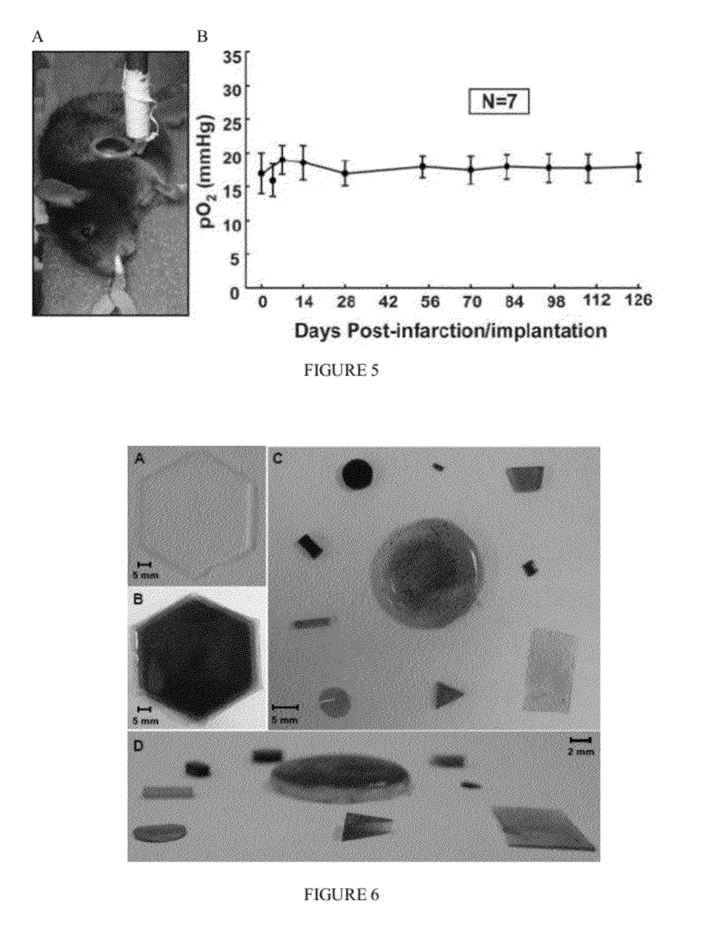

Sensory elements were prepared utilizing the technique of polymerization and cast-molding. For example, a polydimethylsiloxane (PDMS) thin film doped with LiNc-BuO radical microcrystals was fabricated from Dow Corning (Midland, Mich.) medical grade Silastic MDX4-4210 material mixed at recommended base-catalyst ratios. The base-catalyst/LiNc-BuO radical microcrystal mixture was mixed thoroughly, degassed with a vacuum pump, poured into a plastic Petri dish, and allowed to cure by polymerization in an oven at 70° C. for 5-7 h. Small-sized pieces were cut from the cured PDMS thin film for EPR measurements (FIG. 6).

Dose Effect:

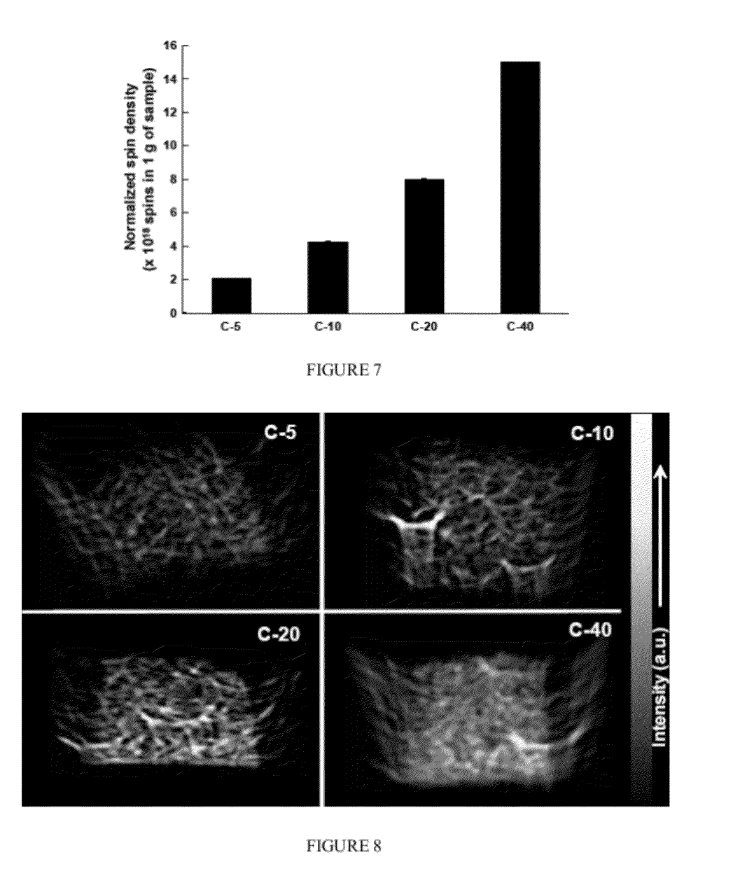

Sensory elements with increasing amounts of LiNc-BuO radical for the same amount of PDMS polymer (5 g) were fabricated. EPR spectra at different controlled oxygen environments were obtained for each of the formulations and calibration curves were constructed (FIG. 7). The sensitivity and linearity of oxygen calibration is not affected within the range of selected LiNc-BuO radical to PDMS polymer ratios (5-40 mg LiNc-BuO to 5 g PDMS polymer) and there is no broadening effect due to increased number of spins (data not shown).

Spin Distribution:

EPR imaging of the sensory elements suggested that LiNc-BuO spins are evenly distributed within the PDMS matrix (FIG. 8). The unusually bright regions (especially in the image corresponding to C-10) may be due to clumping of LiNc-BuO spins at that location during the curing process, leading to a breach in the uniformity of spin distribution. This could be avoided by careful and thorough dispersion of the LiNc-BuO radical microcrystals in the PDMS base-catalyst mixture before it is allowed to cure.

Oxygen Calibration:

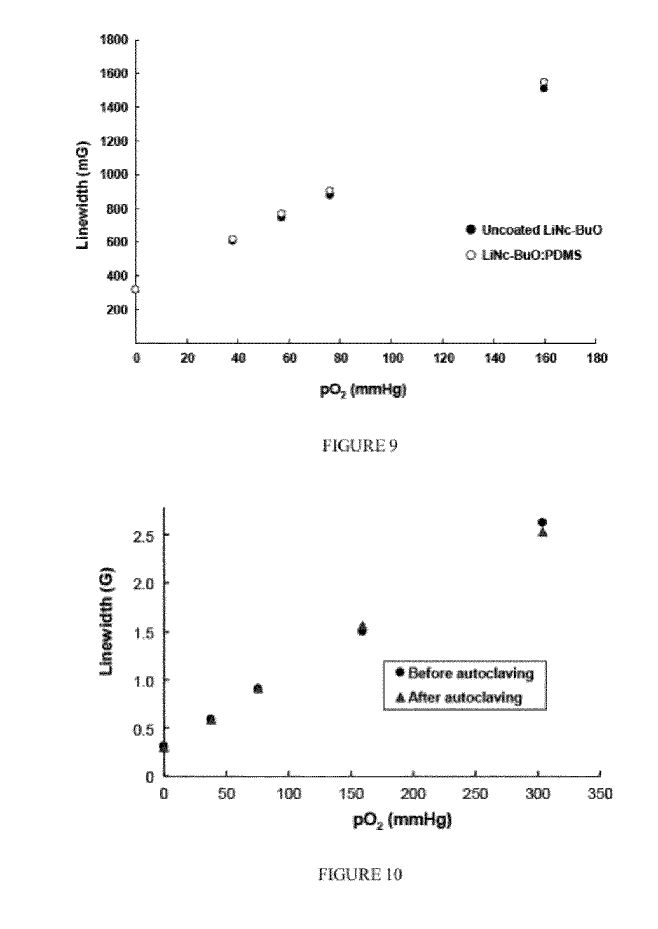

The sensory element responded to changes in oxygen concentration quickly and reproducibly (data not shown), thus enabling dynamic measurements of oxygen in real time. The effect of molecular oxygen concentration (pO₂) on the EPR linewidth of the sensory element (PDMS and LiNc-BuO radical) was determined, and is seen in FIG. 9. The oxygen response of the sensory element was linear over the range of oxygen partial pressure employed (0 to 160 mmHg) for all four sensory element formulations. Responses were very similar to the response exhibited by unencapsulated LiNc-BuO radical microcrystals. The effect of increasing oxygen concentration on spectral linewidth of the LiNc-BuO:PDMS sensory element was highly reversible and reproducible, similar to uncoated LiNc-BuO radical microcrystals. In addition, the oxygen sensitivity (i.e., the slope of calibration curve) of the LiNc-BuO:PDMS sensory element was 7.65±0.07 mG/mmHg, which is not significantly different from the sensitivity of the unencapsulated LiNc-BuO radical microcrystals (7.54±0.10 mG/mmHg).

Effect of Sterilization:

The effect of sterilization treatments, namely autoclaving and ethanol treatment, on sensory elements comprising lithium naphthalocyanine (LiNc) radical and PDMS was evaluated. Autoclaving was performed in a standard bench-top autoclave unit at 121° C. for 1 h at 1 atm pressure (wet cycle using steam) followed by exhaust drying for 15-20 minutes. Also, 70% ethanol was used to sterilize the sensory elements. Samples of the sensory element were soaked in 70% ethanol for 60 min, followed by drying in air for 60 minutes. FTIR spectra collected after the treatments did not show any notable difference in the peak profile, which consisted of characteristic silicone peaks. The FTIR measurements showed that either sterilization treatment does not affect the surface composition of the polymer matrix of the LiNc/PDMS sensory element (data not shown). EPR spectroscopy was used to obtain calibration curves before and after sterilization. A comparison of these calibration curves, seen in FIG. 10, shows that the oxygen calibration of the sensory element remained intact after the sterilization procedure.

Effect of gamma irradiation: Sensory elements were exposed to clinically-relevant doses of gamma radiation (15 & 30 Gy). Effects of the irradiation on spin density and oxygen-calibration were evaluated using EPR spectroscopy. A comparison of the active spin density before and after exposure to the two selected doses of gamma radiation, as seen in FIG. 11, shows no significant change in the active spin content of the sensory elements. Similarly, a comparison of the oxygen calibration, also seen in FIG. 11, reveals that the sensitivity and linearity of the oxygen response of the sensory element is not affected by gamma irradiation.

Long-Term Stability and Response of LiNc-PDMS Chip to Oxygen, In Vivo.

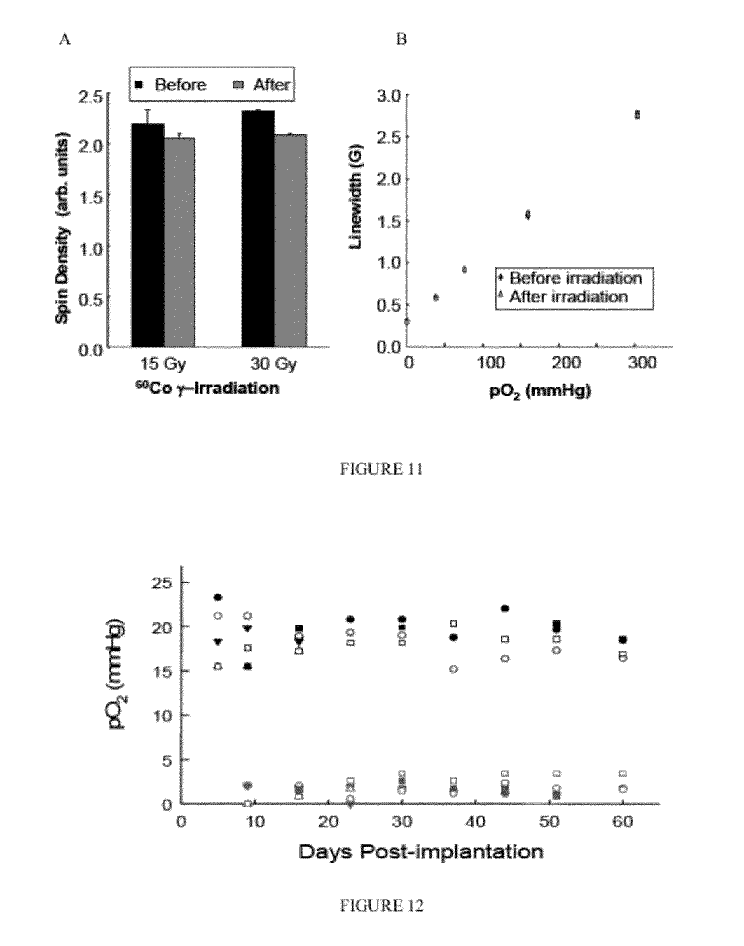

The sensory element comprising LiNc and PDMS was tested for in vivo measurements. The stability of the sensory element sensitivity to oxygen for two months of residence in vivo was studied. The oxygen-response of the LiNc/PDMS sensory element implanted in the leg muscle of C3H mice was monitored up to 60 days. FIG. 12 shows repeated measurements of oxygen concentration from a group of 4 animals. The measurements were performed using an L-Band EPR spectrometer. Linewidth values were converted to oxygen concentration using a calibration curve obtained using a sensory element produced from the same batch as the implanted sensory elements. As seen in FIG. 12, the oxygen response of the implanted sensory element is stable in vivo for a period of two months, or possibly longer.

Stability of EPR and Oxygen Sensitivity for Continuous Monitoring of pO₂.

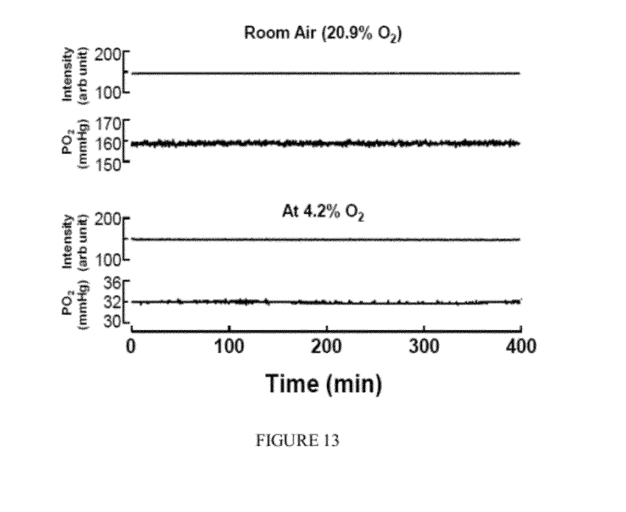

The stability of the sensory element sensitivity and oxygen concentration (pO₂) measurements for continuous monitoring of oxygen concentration were tested. The EPR intensity and pO₂ values were continuously measured for 600 min, while the sensory element was exposed to room air (20.9% oxygen or 159 mmHg) or 4.2% oxygen (32 mmHg). As seen in FIG. 13, the results demonstrate that both the signal intensity and pO₂ readings were not changed during the entire period, suggesting that the sensory element can be used for continuous measurements of oxygen concentration in tissues.

Temporal Transcutaneous Oximetry Data Acquisition.

A sensory element was placed on the right hands of human volunteers. The sensory element was covered with a biocompatible oxygen impermeable material, and the volunteer was asked to place his/her hand under the resonator of a commercially-available L-band EPR unit (Magnettech) for transcutaneous oximetry measurements. The EPR system was adjusted for frequency, tuned accordingly, and 2-3 sample EPR oximetry scans of 10 seconds duration were obtained to maximize signal acquisition and make adjustments to data acquisition parameters. When complete, the volunteer was asked to remain as motionless as possible, as 5 sequential EPR oximetry scans of 10 seconds each were obtained. The volunteer was then allowed to remove his/her hand from the EPR unit and relax. This process was repeated for each data set collected, which was done at 10-minute intervals for the first 90 minutes, with 2 subsequent measurements at 3 hours and 4 hours post application of the sensory element. The EPR measurement data was analyzed using a curve-fitting program to obtain transcutaneous oxygen partial pressure (pO₂) values. These values were then plotted temporally using time-stamp data collected during data acquisition. A regression line was then fitted to the data. The type of regression line applied was selected based upon the returned R-squared (R2) value, which indicates quality of fit (greater is better).

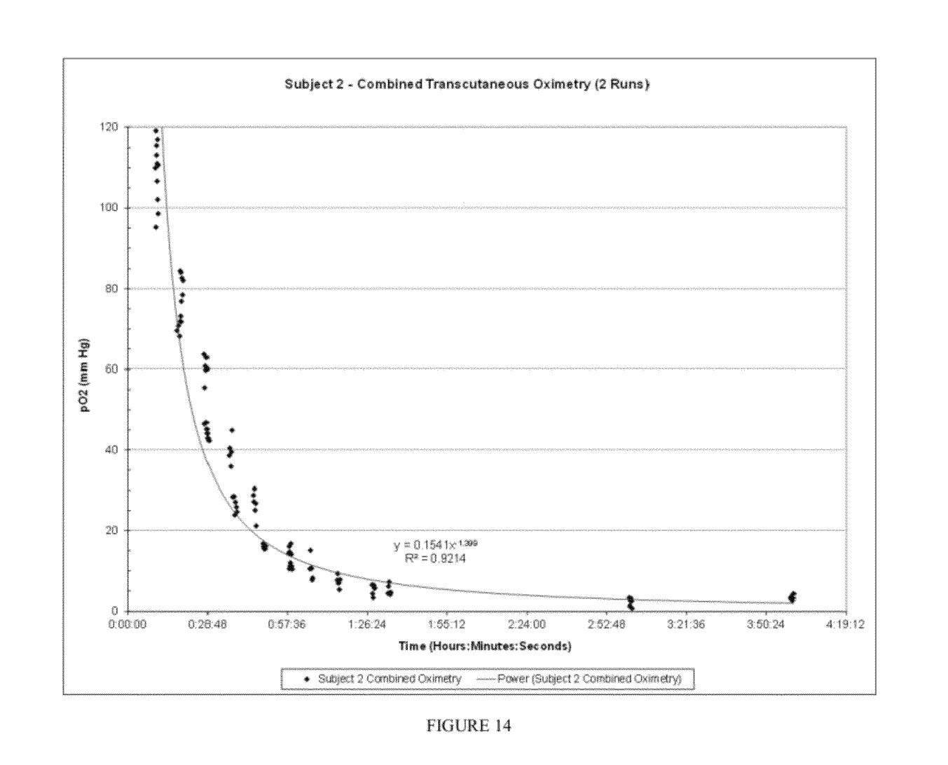

As shown in FIG. 14, the temporal response profile of the sensory element follows an exponential decay curve. After approximately 90 minutes, the sensory element appears to reach an equilibrium perfusion condition, as there is little variation in the pO₂ measurements from 90 minutes to 3 hours to 4 hours after application of the sensory element. The measurements were consistent and repeatable. The lag-time to reach a steady-state perfusion condition likely represents oxygen that is “trapped” within the sensory element during application, as this was done in room air and not under vacuum or anoxic conditions.

Transcutaneous Oximetry Data Acquisition from the Skin of a Human Subject.

A sensory element was placed on the right hand of a human volunteer. The sensory element was covered with a biocompatible oxygen impermeable material, and allowed 30 minutes to equilibrate. After this period, the volunteer was asked to place his/her hand under the resonator of a commercially-available L-band EPR unit (Magnettech) for baseline transcutaneous oximetry measurements.

The EPR system was adjusted for frequency, tuned accordingly, and 2-3 sample EPR oximetry scans of 10 seconds duration were obtained to maximize signal acquisition and make adjustments to data acquisition parameters. When complete, the volunteer was asked to remain as motionless as possible, as 5 sequential EPR oximetry scans of 10 seconds each were obtained. The volunteer was then allowed to remove his/her hand from the EPR unit and relax. This same process was repeated for each data set collected.

Upon completion of the baseline scans, a standard phlebotomy tourniquet was tightened and secured around the arm of the volunteer, just above the elbow, restricting bloodflow to the hand with the attached sensory element. The volunteer's hand was again placed in the EPR unit, and transcutaneous oximetry scans were obtained as before at 1 minute post-tourniquet application. The tourniquet was left in place, and the process was repeated at 10 minutes post-tourniquet application. The tourniquet was subsequently removed, and EPR measurements were obtained yet again upon reperfusion at 3 minutes post-removal of the tourniquet, and again at 10 minutes post-removal of the tourniquet. The EPR measurement data was analyzed using a curve-fitting program to obtain transcutaneous oxygen partial pressure (pO₂) values.

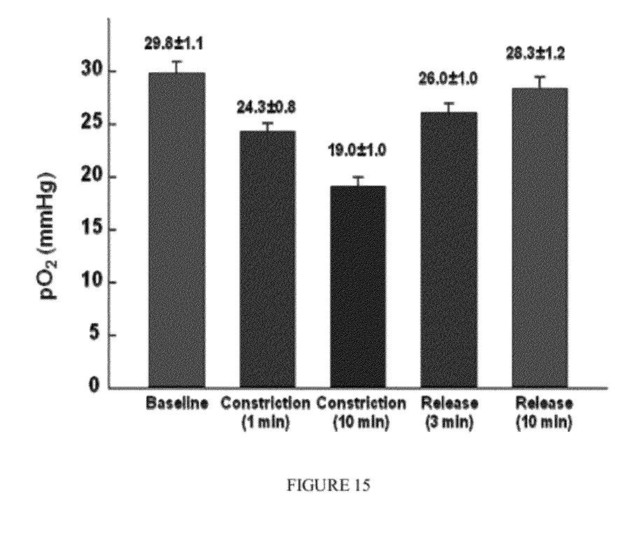

The results of the oximetry measurement data analysis are shown in FIG. 15. The baseline transcutaneous oximetry measurements produced a oxygen partial pressure oxygen (pO₂) value of 29.8±1.1 mm Hg. Upon application of the tourniquet, the pO₂ decreased to 24.3±0.8 mm Hg at 1 minute post-tourniquet application, and dropped further to 19.0±1.0 mm Hg at 10 minutes post-tourniquet application. Subsequent removal of the tourniquet produced a recovery in transcutaneous pO₂ to 26.0±1.0 mmHg at 3 minutes post-removal, and 28.3±1.2 mmHg at 10 minutes post-release. The results demonstrate that the sensory element was capable of detecting and allowing quantification (in terms of tissue oxygenation) of a decrease in transcutaneous oxygen perfusion, followed by recovery, in a human volunteer. This demonstrates the feasibility of using the sensors for real-time pO₂ monitoring on human subjects.

What is claimed is:

1. A sensor for measuring oxygen concentration in a tissue or an organ of a subject, comprising:
a sensory element comprising at least one paramagnetic spin probe compound encapsulated in a biocompatible oxygen permeable material; and
a barrier layer partially covering the sensory element, the barrier layer comprising at least one biocompatible oxygen impermeable material;
wherein the sensory element includes a sensory contact surface for contacting the tissue or the organ of the subject, and the barrier layer covers the outer surface of the sensory element except for the sensory contact surface.

2. The sensor according to claim 1, wherein the at least one paramagnetic spin probe compound is selected from the group consisting of 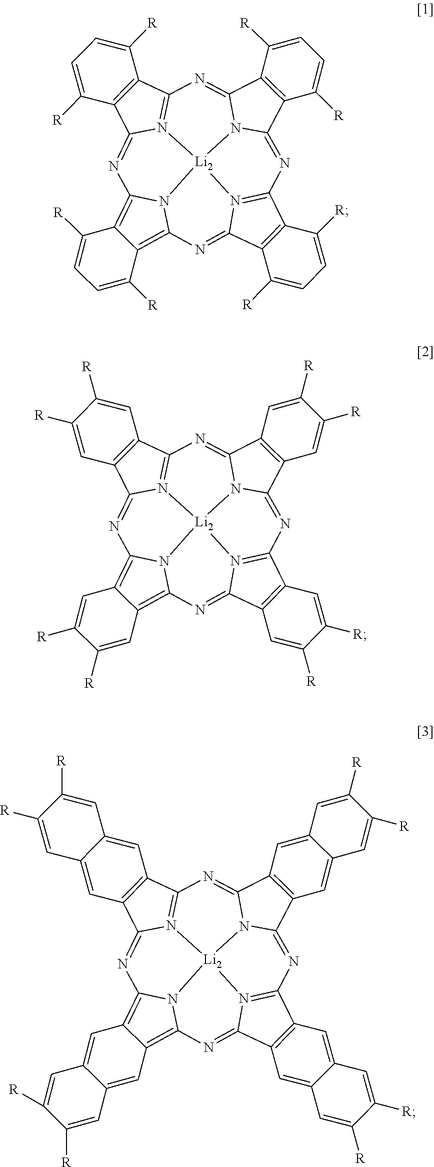 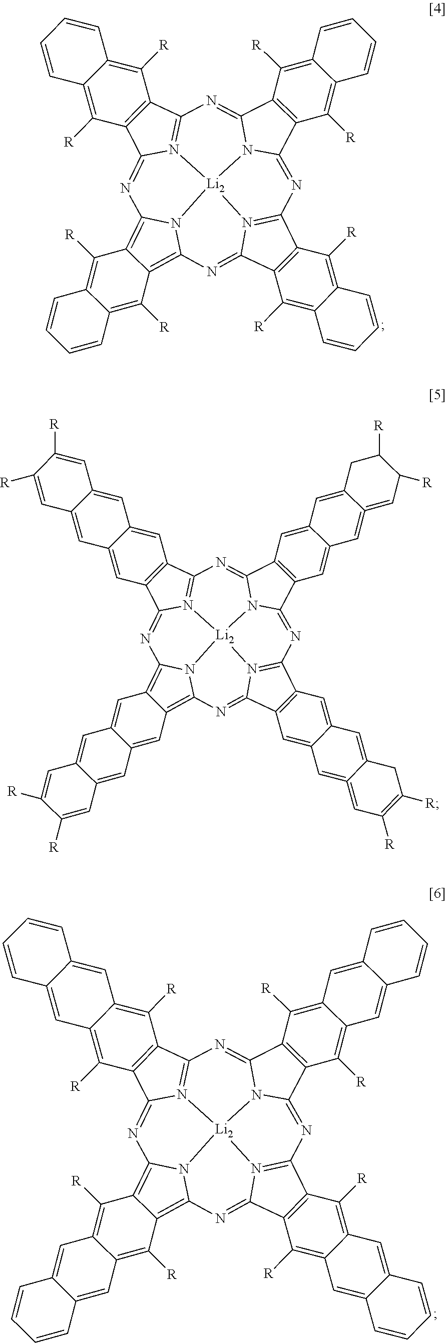
and radicals thereof, wherein R is selected from the group consisting of H, O(CH₂)ₙCH₃, S(CH₂)ₙCH₃, O(CH₂)ₙCH₂OH, O(CH₂)ₙCH₂NH₂, O(CH₂)ₙCH₂SH, and combinations thereof, wherein n is 1-6, and combinations thereof.

3. The sensor according to claim 1, wherein the biocompatible oxygen permeable material is selected from the group consisting of polydimethylsiloxane, an amorphous fluoropolymer, fluorosilicone acrylate, cellulose acetate, polyvinyl acetate, and combinations thereof.

4. The sensor according to claim 1, wherein the at least one biocompatible oxygen impermeable material is selected from the group consisting of polyvinyl alcohol, a poly(p-xylylene) polymer, an aluminum oxide-coated polyester film, a polyacrylic acid-coated polyester film, ethylene-vinyl alcohol, and combinations thereof.

5. The sensor according to claim 1, further including an adhesive layer on at least a portion of the outer surface of the barrier layer.

6. The sensor according to claim 2, wherein the at least one paramagnetic spin probe comprises 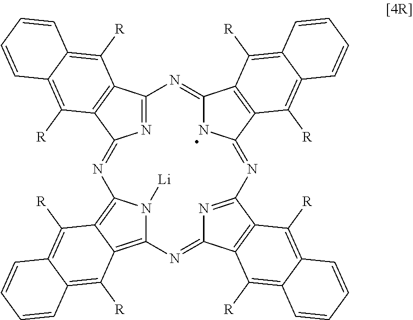
wherein R is O(CH₂)ₙCH₃, and n is equal to 3;
the biocompatible oxygen permeable material is polydimethylsiloxane; and
the at least one biocompatible oxygen impermeable material is polyvinyl alcohol.

7. The sensor according to claim 1, wherein the sensory element further includes an outer protective layer comprised of at least one biocompatible oxygen permeable material.

8. The sensor according to claim 7, wherein the outer protective layer is comprised of polydimethylsiloxane.

9. The sensor according to claim 1, wherein the sensor has a thickness of about 0.50 millimeter to about 1.00 centimeter.

10. The sensor according to claim 9, wherein the sensory element has a thickness of about 0.25 millimeter to about 5.00 millimeter, and the barrier layer has a thickness within a range of about 0.25 millimeter to about 1.00 centimeter.

11. The sensor according to claim 1, wherein the weight ratio of the at least one paramagnetic spin probe compound to the biocompatible oxygen permeable material is within a range of 1:1000 to 1:125.

12. A method of measuring oxygen concentration in a tissue or an organ of a subject, the method comprising the steps of:
a) applying a sensor according to claim 1 to the tissue or the organ of the subject; and
b) applying a magnetic resonance spectroscopy or imaging technique to obtain data corresponding to the concentration of oxygen present in the tissue or the organ of the subject.

13. The method of claim 12, wherein the magnetic resonance spectroscopy or imaging technique is selected from the group consisting of electron paramagnetic resonance, electron spin resonance, electron paramagnetic resonance imaging, magnetic resonance imaging, and proton-electron double-resonance imaging.

14. The method of claim 13, wherein the magnetic resonance spectroscopy or imaging technique is electron paramagnetic resonance.

15. The method according to claim 12, wherein after step a), the method further comprises the step of waiting a predetermined amount of time before proceeding to step b).

16. The method according to claim 15, wherein the predetermined amount of time is from about 5 minutes to about 90 minutes.

17. The method according to claim 12, wherein the tissue or the organ of the subject is the skin, and the sensor is directly applied to the skin of the subject.

18. The method according to claim 17, wherein the subject is a human.
